(19) United States
(12) Patent Application Publication
(10) Pub. No.: US 2009/0074944 A1
(43) Pub. Date: Mar. 19, 2009

(54) ENTERIC COATINGS FOR ORALLY INGESTIBLE COMPOSITIONS

(75) Inventors: Xueju XIE, Richmond (CA); Edward Ko, Richmond (CA); David Ko, Richmond (CA); Jason Jiang-Chung Ho, Richmond (CA)

Correspondence Address: FASKEN MARTINEAU DUMOULIN, LLP, 2900 - 550 Burrard Street, VANCOUVER, BC, V6C 0A3, CA

(73) Assignee: VIVA PHARMACEUTICALS INC., Richmond (CA)

(21) Appl. No.: 12/019,384

(22) Filed: Jan. 24, 2008

Related U.S. Application Data
(60) Provisional application No. 60/973,435, filed on Sep. 18, 2007.

(51) Int. Cl. A61K 9/48; C08L 1/08

(52) U.S. Cl. 427/2.14; 106/217.7

(57) ABSTRACT

A suspendible enteric coating composition for encasing orally ingestible articles wherein the enteric coating composition comprises a pH-dependent polymer selected from a group containing alginates and alginic acids, a pH-independent water insoluble polymer selected from the group comprising ethylcellulose and ethylcellulose-containing compositions, and a plasticizer selected from the group containing triethyl citrate, glycerin, propylene glycol, triacetin, acetylated monoglycerides, dibutyl sebacate, polyethylene glycols, sorbitals, middle chain triglycerides and combinations thereof. A three step method for providing a stable outer enteric coating on an ingestable item comprising a first step of encasing the item with a suspension comprising a mixture of at least a sugar and a microcrystalline cellulose, a second step of then encasing the item with a suspension comprising a mixture of a film-forming polymer and a plasticizer, and a third step of finally encasing the item with the enteric coating composition.

### FIELD OF THE INVENTION

This invention relates to enteric coatings for encapsulated orally ingestible compositions exemplified by pharmaceutical compositions, nutraceutical compositions, nutritional supplements, foodstuffs and the like. More particularly, this invention relates to enteric coating compositions and to methods for applying said enteric coating compositions.

### BACKGROUND OF THE INVENTION

There are many enteric coating materials currently available for use as outer coatings on capsules and tablet formulations containing chemically stable pharmaceutical compositions. Examples of such enteric coating materials include Aquacoat® CPD (Aquacoat is a registered trademark of the FMC Corporation), Eudragit® methacrylic copolymers (Eudragit is a registered trademark of Rohm & Haas G.M.B.H. Co.), Kollicoat® MAE (Kollicoat is a registered trademark of the BASF Aktiengesellschaft Corp.), Acryl-EZE® (Acryl-EZE is a registered trademark of BPSI Holdings Inc.), Opadry® (Opadry is a registered trademark of BPSI Holdings Inc.), and Sureteric® (Sureteric is a registered trademark of BPSI Holdings Inc.). Each of the afore-mentioned enteric coating materials is a proprietary formulation. However, there has been an emergence of significant market and consumer demands for novel chemical and biological-based pharmaceutical, nutraceutical and nutritional supplement compositions that are less stable and therefore, present new challenges for enteric coating materials with regard to: (a) post-manufacture chemical compatibility, stability and storage properties, (b) post-ingestion functionality, and (c) the replacement of organic synthesized components with components derived from naturally occurring materials. Consequently, there is a need for novel enteric coating materials, compositions, and coating methods that are compatible with less stable pharmaceutical compositions, nutraceutical compositions, nutritional supplement compositions, and foodstuffs.

Sodium alginate is a sodium salt of alginic acid. Alginic acid is a naturally occurring linear copolymer with homopolymeric blocks of (1-4)-linked β-D-mannuronate (M) and its C-5 epimer α-L-guluronate (G) residues, respectively, covalently linked together in different sequences or blocks. Sodium alginate is soluble in water having a pH of 4 and greater. However, in acidic environments having pH values less than 4, e.g., in gastrointestinal systems, sodium alginate is converted into the insoluble alginic acid form. The physico-chemical properties of sodium alginate are well-known and have been exploited for many food and pharmaceutical applications as supplementary thickeners, coating materials and in the formulation of controlled-release compositions.

Grillo et al (U.S. Pat. No. 6,468,561) teach the use of polydextrose or a combination of polydextrose and other polymers to enhance adhesive quality and color stability of tablet-coating films. Grillo et al also provide formulations comprising 30 to 90% of polydextrose (w/w). Polydextrose is very soluble in water regardless of the pH values. Therefore, if such high concentrations of polydextrose were incorporated into enteric coating formulations, the coating functions and stabilities of the coated articles would be significantly impaired after their oral ingestion because polydextrose in the coating film will be rapidly dissolved. The consequence would be rapid breakdown and disintegration of the coating thereby resulting in premature release of the constituents of the coated articles into the stomach contents. Grillo et al also teach that overcoating their polydextrose coating with a secondary coating comprising 2% to 10% sodium alginate (w/w) may enhance the adhesiveness and color stability of their films.

Zhang et al (U.S. Pat. No. 6,251,430) teach a sustained-release drug dosage tablet formulation comprising a combination of: (a) a mixture of a water-insoluble polymer, a pH-dependent gelling polymer, a pH-independent gelling polymer, and (b) an active ingredient that requires a time-release profile. Zhang et al's formulation requires the three polymers, i.e., the water-insoluble polymer, the pH-dependent gelling polymer and the pH-independent gelling polymer, to be commingled in order to provide controlled-release of the intermixed active ingredient. Their water-insoluble polymer was exemplified by ethylcellulose and copolymers of acrylic and methacrylic acid esters (e.g., Eudragit®). Their pH-dependent gelling polymer was exemplified by alginates and sodium carboxymethylcellulose. Their pH-independent gelling polymer was exemplified by hydroxypropyl cellulose, hydroxypropyl methylcellulose, hydroxypropyl ethylcellulose, carboxypolymethylene and the like. Zhang et al's tablet-compressing system is not applicable for soft-capsule systems because soft-capsules are formed by injection methods that require the active ingredients to be provided in a liquid or paste form.

Kim et al. (U.S. Pat. No. 6,365,148) teach the preparation of granulated microbial inocula by spray-coating bacteria in a fluidized bed granulator with a water-miscible coating composition thereby producing granules wherein each granule comprises a plurality of bacteria encased in the water-miscible composition. Kim et al teach that their water-miscible coating composition may comprise one or more of alginates, gums, wheat proteins, methylcellulose, hydroxypropyl cellulose, hydroxypropyl methylcellulose, polyvinylacetate phthalate and the like. Using Zhang et al's terminology, Kim et al's water-miscible coating comprises one or more of a pH-dependent gelling polymer and/or a pH-independent gelling polymer. Kim et al. further teach an optional second coating for application as an overcoat to their granulated microbial inocola, wherein the second coating is a controlled-release composition.

### SUMMARY OF THE INVENTION

The exemplary embodiments of the present invention, are directed to suspendible enteric coating compositions configured for encasing orally ingested articles, and to methods for applying suspendible enteric coating compositions.

An exemplary embodiment of the present invention is directed to enteric coating compositions suspendible in water for encasing therewith orally ingestible articles exemplified by soft-gel capsules, hard-gel capsules, tablets, pellets and the like. The enteric coating compositions comprise at least a pH-dependent polymer, a pH-independent water insoluble polymer, and a plasticizer.

According to one aspect, the pH-dependent polymer is selected from a group containing alginates and alginic acids.

According to another aspect, the pH-independent water insoluble polymer is selected from the group containing ethylcellulose and ethylcellulose-containing compositions.

According to another aspect, the plasticizer is selected from the group containing triethyl citrate, glycerin, propylenglycol, triacetin, acetylated monoglycerides, dibutyl sebacate, polyethyleneglycols and middle chain triglycerides.

According to a further aspect, the enteric coating compositions may optionally comprise at least one of a flavorant and/or a colorant.

Another exemplary embodiment of the present invention is directed to a three-step method for coating mated two-piece hard-shell capsules. The first step generally comprises encasing a plurality of mated two-piece hard-shell capsules with a first suspension comprising (1) a mixture of at least a sugar and a microcrystalline cellulose, or (2) the combination of at least two of the materials selected from the group comprising modified starch, maltodextrin, dextrin, microcrystalline cellulose, carboxylmethylcellulose (CMC) and polysaccharides, and then drying said mated two-piece hard-shell capsules thereby providing a first coat thereon. The second step generally comprises encasing a plurality of the first-coated mated two-piece hard-shell capsules with a second suspension comprising a mixture of a film-forming polymer and a plasticizer, and then drying the mated two-piece hard-shell capsules to providing a second coat encasing the first coat. The film-forming polymer may be optionally mixed with a gel-forming agent and a plasticizer. The third step generally comprises encasing a plurality of the second-coated mated two-piece hard-shell capsules with a third suspension comprising a selected enteric coating, and then drying hard-shell capsules to provide a third coat encasing the first and second coats.

Another exemplary embodiment of the present invention is directed to a two-step method for coating mated two-piece hard-shell capsules. The first step generally comprises encasing a mated two-piece hard-shell capsules with a first suspension comprising a mixture of: (1) maltodextrin or polydextrose or both, (2) at least one ingredient selected from the group of modified starch, microcrystalline cellulose hydroxypropylene methylcellulose (HPMC), carboxylmethylcellulose (CMC) and polysaccharides. The first suspension may additionally comprise (3) a plasticizer selected from the group comprising triethyl citrate, glycerin, propylenglycol, triacetin, acetylated monoglycerides, dibutyl sebacate, polyethyleneglycols and middle chain triglycerides. After receiving a coating with the first suspension, the mated two-piece hard-shell capsules are then dried. The second step generally comprises encasing the first-coated mated two-piece hard-shell capsules with a second suspensions comprising a selected enteric coating, and then drying hard-shell capsules to provide a second coat encasing the first coat.

### DETAILED DESCRIPTION OF THE INVENTION

The present invention is directed to enteric coating compositions suitable for orally ingestible articles exemplified by soft-gel capsules, hard-shell capsules, tablets and the like, and to methods for the application of the enteric coatings onto the orally ingestible articles.

### EXAMPLE 1

### Enteric Coating Compositions for Orally Ingestible Articles Exemplified by Soft-Gel Capsules, Hard-Shell Capsules, Tablets, Pellets and the Like

The present invention provides an exemplary enteric coating wherein the coating comprises three components. The first component is a pH-dependent polymer that is: (a) soluble in solutions having a pH value of 4, and (b) insoluble in solutions having a pH value less than 4. Suitable pH-dependent polymers are exemplified by sodium alginate, alginic acid and the like.

The second component is a pH-independent water insoluble polymer exemplified by ethylcellulose. Suitable commercially available ethylcelluloses are exemplified by Aquacoat® ACD that comprises 30% ethylcellulose (w/w) (supplied by FMC BioPolymer, 1735 Market Street, Philadelphia, Pa., 19103, USA) and Surelease® that comprises 25% ethylcellulose (supplied by Colorcon, Inc., 420 Moyer Blvd., West Point, Pa., 19486, USA).

The third component is a plasticizer exemplified by triethyl citrate, glycerin, propylenglycol, triacetin, acetylated monoglycerides, dibutyl sebacate, polyethyleneglycols, sorbitol, sorbitol special, middle chain triglycerides, and the combinations thereof.

An exemplary enteric coating composition according to the present invention, in a dry form, comprises: (a) about 10% to about 20% of a pH-dependent polymer, (b) about 40% to 75% of a pH-independent water insoluble polymer, and (c) about 7% to about 20% of a suitable plasticizer. The dry form of the enteric coating composition may be suspended in a suitable solvent exemplified by water, to product an enteric coating suspension which will comprise: (a) about 1% to about 2% of the suitable pH-dependent polymer, (b) about 5% to about 10% of the suitable pH-independent water insoluble polymer, and (c) about 0% to 3% of the suitable plasticizer.

It is within the scope of the present invention to incorporate suitable colorants into the enteric coating compositions described herein. Suitable colorants are exemplified by dyes, titanium dioxide, iron oxides, natural pigments, pearlescent pigments or other pigments approved by regulatory agencies such as the USDA, FDA and Health Canada among others.

It is also within the scope of the present invention to incorporate suitable flavorants into the enteric coating compositions described herein. Suitable flavorants are exemplified by those that are currently approved by regulatory agencies such as the USDA, FDA and Health Canada among others.

It should be noted that the enteric coating compositions disclosed here in are suitable for application onto soft-gel capsules, hard-gel capsules, tablets, pellets, granules and the like containing therein orally ingestible components.

### EXAMPLE 2

### An Exemplary Enteric Coating Composition

| Component | Tradename | Weight |
|---|---|---|
| pH-dependent polymer | Sodium alginate | 1.5 |
| pH-independent water insoluble | Aquacoat ECD | 28.3 |
| polymer | (30% ethylcellulose) | |
| Plasticizer | Triethyl citrate | 2.1 |
| Distilled water | water | 68.1 |

The three components were mixed into the water at room temperature until fully suspended. The enteric coating suspension thus produced was coated onto pre-weighed softgels, allowed to dry, after which, the coated softgels were re-weighed. The coated softgels weighed about 9.5% more than the uncoated softgels. The coated softgels were then placed into a low pH gastric fluid solution (pH˜2) to determine coating stability in pH and enzyme conditions that approximate stomach acidity conditions, and then, were removed from the low pH solution and transferred to a neutral pH intestinal fluid solution (pH˜6) to determine coated softgel disintegration in pH conditions that approximate intestinal fluid conditions. No visible disintegration was detectable after 60 minutes in the low pH solution. However, the coating completely disintegrated within 60 minutes in the neutral pH solution.

### EXAMPLE 3

### Enteric Coating Compositions for Orally Ingestible Articles Exemplified by Soft-Gel Capsules, Hard-Shell Capsules, Tablets, Pellets and the Like

Orally ingestible hard-shell capsules are known to be particularly difficult to provide satisfactory enteric coatings onto. Hard-shell capsules generally comprise a bottom half-capsule matable to a top half-capsule. The bottom half-capsule is generally configured for receiving therein active ingredients to be encapsulate, while the top half-capsule is generally configured for continuously contacting the outer edges of the bottom half-capsule for containing therein the active ingredients. However, before the filled and mated hard capsule configuration can be coated with an enteric coating composition, the outer surfaces of the top half-capsule and bottom half-capsule have to be pre-coated with an elastic film-forming material. It is essential that the elastic film pre-coat is sufficiently thick to fill the juncture seam between the top half-capsule and the bottom half-capsule and provide a smooth continuous surface about and around the two mated half-capsules. Furthermore, it is desirable that the elastic film pre-coat is sufficiently flexible and pliable to absorb mechanical stresses and pressures during the coating processes while sealably containing the mated half-capsules. Consequently, the initial elastic film pre-coating step is time-consuming and critical for satisfactory subsequent application of the enteric coatings.

Another exemplary embodiment of the present invention is directed to a three-step method for application of suitable enteric coatings to hard-shell capsules that overcomes the current problems commonly encountered in providing suitable enteric coatings onto hard-shell capsules. The first step generally comprises applying to a mated hard-shell capsule, an encasing first coating of an aqueous solution comprising about 40% of solids including microcrystallinecellulose and sucrose exemplified by LustreSugar® (LustreSugar is a registered trademark of the FMC Corporation) so that the weight of the mated hard-shell capsule is increased by about 15% after the first coating has dried. It only took one and a half hour to coat because of high solid content solution. The first coating provides sealing and binding for holding the two half-capsules together. The second step generally comprises applying to the once-coated hard-shell capsule, a second encasing coating solution comprising a mixture of at least a film-forming polymer and/or a gel-forming agent. A suitable film-forming agent is exemplified by microcrystalline cellulose, hydroxypropylene methylcellulose (HPMC), hydroxypropylcellulose (HPC) or other available film-forming polymers. A suitable gel-forming agent is exemplified by polysaccharides, such as carrageenan and alginates, carboxymethylcellulose. An exemplary suitable commercial preparation containing a suitable firm-forming agent and a suitable gel-forming agent is LustreClear® (LustreClear is a registered trademark of the FMC Corporation). An exemplary second encasing solution is an aqueous suspension containing 10% LustreClear®. The second encasing suspension is applied to the once-coated hard-shell capsule so that the weight of the once-coated mated hard-shell capsule is increased by about 7 to 15% after the second coating has dried. The third step generally comprises applying to the twice-coated mated hard-shell capsule, an encasing coating of a suitable enteric coating. Suitable enteric coatings are exemplified by Aquacoat® CPD, Eudragit® methacrylic copolymers, Kollicoat® MAE, Surelease®, Acryl-EZE®, Opadry®, Sureteric®, and the like. It is within the scope of the present invention to optionally incorporate flavorants and/or colorants into one or more the coatings applied in the present 3-step coating method.

### EXAMPLE 4

### An Exemplary 3-Step Enteric Coating Method for Hard-Shell Capsule

The first step comprised preparation of an aqueous suspension comprising 40% Lustre Sugar® dissolved in distilled water. Mated hard-shell capsules were then first coated with the Lustre Sugar® solution and then dried. The dry weight of the first-coated mated hard-shell capsules increased by 15% of the weight of the mated hard-shell capsules.

The second step comprised preparation of an aqueous suspension comprising 10% LustreClear® LC-103, 5% of glycerin (plasticizer), and 85% distilled water. The dried first-coated mated hard-shell capsules were then encapsulatingly coated a second time using the LustreClear® suspension and then were dried. The dry weight of the second-coated mated hard-shell capsules increased by 15% over the weight of the first-coated mated hard-shell capsules.

The third step comprised preparation of an enteric coating suspension comprising 10% Kollicoat® MAE-100P, 5% propylene glycol, 85% distilled water. The dried second-coated mated hard-shell capsules were then encapsulatingly coated a third time using the Kollicoat® suspension and then were dried. The dry weight of the third-coated mated hard-shell capsules increased by 9% over the weight of the second-coated mated hard-shell capsules. After drying, the third-coated mated hard-shell capsules possessed a very smooth and seamless opaque outer coating.

### EXAMPLE 5

### An Exemplary 2-Step Enteric Coating Method for Hard-Shell Capsules

Another exemplary embodiment of the present invention is directed to a two-step method for application of suitable enteric coatings to hard-shell capsules that overcomes the current problems commonly encountered in providing suitable enteric coatings onto hard-shell capsules. The first step comprises applying to a mated hard-shell capsule, an encasing first coating of an aqueous solution comprising about 50% of solids including 20% polydextrose, 20% maltodextrin and 10% starch 1500. The first-coated mated hard-shell capsules increased by 13% of the weight of the mated hard-shell capsules. The dried first-coated mated hard-shell capsules, which gave a transparent appearance, were then encapsulatingly coated a second time using the Kollicoat® suspension and then were dried. The dry weight of the second-coated mated hard-shell capsules increased by 9% over the weight of the second-coated mated hard-shell capsules. After drying, the second-coated mated hard-shell capsules possessed a transparent appearance. The coated softgels were then placed into a low pH gastric fluid solution (pH˜2) to determine coating stability in pH and enzyme conditions that approximate stomach acidity conditions, and then, were removed from the low pH solution and transferred to a neutral pH intestinal fluid solution (pH˜6) to determine coated softgel disintegration in pH conditions that approximate intestinal fluid conditions. No visible change in shape was detectable after 60 minutes in the low pH solution. However, the coating completely disintegrated within 60 minutes in the neutral pH solution.

### EXAMPLE 6

### An Exemplary 2-Step Enteric Coating Method for Hard-Shell Capsules

The first step comprises applying to a mated hard-shell capsule, an encasing first coating of an aqueous solution comprising about 47% of solids including 18% polydextrose, 18% maltodextrin, 9% instant pure-cote B793 (starch) and 2% glycerin. The first-coated mated hard-shell capsules increased by 20% of the weight of the mated hard-shell capsules. The dried first-coated mated hard-shell capsules were then encapsulatingly coated a second time using the Kollicoat® suspension and then were dried. The dry weight of the second-coated mated hard-shell capsules increased by 9% over the weight of the second-coated mated hard-shell capsules. After drying, the second-coated mated hard-shell capsules possessed a transparent appearance. The coated softgels were then placed into a low pH gastric fluid solution (pH˜2) to determine coating stability in pH and enzyme conditions that approximate stomach acidity conditions, and then, were removed from the low pH solution and transferred to a neutral pH intestinal fluid solution (pH˜6) to determine coated softgel disintegration in pH conditions that approximate intestinal fluid conditions. No visible change in shape was detectable after 60 minutes in the low pH solution. However, the coating completely disintegrated within 60 minutes in the neutral pH solution.

While this invention has been described with respect to the exemplary embodiments, it is to be understood that various alterations and modifications can be made to the enteric coating compositions, and to methods of applying enteric coating compositions can be made within the scope of this invention, which are limited only by the scope of the appended claims.

We claim:

1. A suspendible enteric coating composition for encasing orally ingestible articles, the enteric coating composition comprising:
a pH-dependent polymer;
a pH-independent water insoluble polymer; and
a plasticizer.

2. An enteric coating composition according to claim 1, wherein the pH-dependent polymer is selected from a group containing alginates and alginic acids.

3. An enteric coating composition according to claim 1, wherein the pH-independent water insoluble polymer is selected from the group containing ethylcellulose and ethylcellulose-containing compositions.

4. An enteric coating composition according to claim 1, wherein the plasticizer is selected from the group containing triethyl citrate, glycerin, propylene glycol, triacetin, acetylated monoglycerides, dibutyl sebacate, polyethylene glycols, sorbitals, middle chain triglycerides and combinations thereof.

5. An enteric coating composition according to claim 1, additionally comprising at least one colorant.

6. An enteric coating composition according to claim 1, additionally comprising at least one flavorant.

7. A three-step method for providing mated two-piece hard-shell capsules with a stable enteric outer coating, the three-step method comprising:
a first step comprising encasing a plurality of mated two-piece hard-shell capsules with a first suspension comprising a mixture of at least a sugar and a microcrystalline cellulose, and then drying said encased mated two-piece hard-shell capsules thereby providing a first encasing coat thereon;
a second step comprising encasing a plurality of first-coated mated two-piece hard-shell capsules with a second suspension comprising a mixture of a film-forming polymer and a plasticizer, and then drying said mated two-piece hard-shell capsules thereby providing a second encasing coat thereon; and
a third step comprising encasing a plurality of second-coated mated two-piece hard-shell capsules with a third suspension comprising an enteric coating composition, and then drying said encased mated two-piece hard-shell capsules thereby providing a third encasing coat thereon, said third encasing coat comprising a stable outer coating.

8. A three-step method according to claim 7, wherein the second suspension comprises a mixture of a film-forming polymer, a gel-forming agent, and a plasticizer.

9. A three-step method according to claim 8, where the second suspension comprises at least one material selected from the group consisting of modified starch, maltodextrin, dextrin, microcrystalline cellulose, carboxymethylcellulose, hydroxyethylcellulose, hydroxypropylene methylcellulose, and polysaccharides,

10. A three-step method according to claim 8, where the second suspension comprises a combination of at least two of the materials selected from the group consisting of modified starch, maltodextrin, dextrin, microcrystalline cellulose, carboxymethylcellulose, hydroxyethylcellulose, hydroxypropylene methylcellulose, and polysaccharides,

11. A three-step method according to claim 7, wherein the second suspension additionally comprises at least one additional component selected from the group containing colorants and flavorants.

12. A three-step method according to claim 7, wherein the third suspension additionally comprises at least one additional component selected from the group containing plasticizers, colorants, and flavorants.

13. A two-step method for providing mated two-piece hard-shell capsules with a stable enteric outer coating, the method comprising:
a first step comprising encasing a plurality of mated two-piece hard-shell capsules with a first suspension comprising a mixture of (a) a selection from the group containing maltodextrin, polydextrose, and combinations thereof, and (b) at least one ingredient selected from the group containing modified starch, microcrystalline cellulose, hydroxypropylene methylcellulose, hydroxylethylcellulose, carboxylmethylcellulose, and polysaccharides, and drying said first suspension onto said mated two-piece hard-shell capsules thereby providing a first encasing coat thereon; and
a second step comprising encasing a second time said mated two-piece hard-shell capsules with a second suspension comprising an enteric coating composition, and then drying said mated two-piece hard-shell capsules thereby providing a second encasing coat thereon, said second encasing coat comprising a stable outer coating.

14. A two-step method according to claim 13, wherein the first suspension additionally comprises a plasticizer selected from the group consisting of triethyl citrate, glycerin, propylenglycol, triacetin, acetylated monoglycerides, dibutyl sebacate, polyethyleneglycols, sorbitols, middle chain triglycerides, and combinations thereof.

15. A two-step method according to claim 13, wherein the first suspension additionally comprises at least one additional component selected from the group containing colorants and flavorants.

16. A two-step method according to claim 13, wherein the second suspension additionally comprises at least one additional component selected from the group containing colorants and flavorants.
